SEQUENCE LISTING (42 sequences)

SEQ ID NO: 1 - DNA, 13 bp, Artificial Sequence
  Description of Artificial Sequence Synthetic oligonucleotide
  modified_base (12)..(12): a, c, t, or g
ccccwccwccvnd

SEQ ID NO: 2 - DNA, 13 bp, Artificial Sequence
  Description of Artificial Sequence Synthetic oligonucleotide
ccccacctccccg

SEQ ID NO: 3 - DNA, 15 bp, Artificial Sequence
  Description of Artificial Sequence Synthetic probe
cgaacccccwccwyc

SEQ ID NO: 4 - DNA, 15 bp, Artificial Sequence
  Description of Artificial Sequence Synthetic probe
cgaacccccacctcc

SEQ ID NO: 5 - DNA, 38 bp, Artificial Sequence
  Description of Artificial Sequence Synthetic probe
  modified_base (27)..(27): a, c, t, or g
  modified_base (32)..(32): a, c, t, or g
caaccccaatcgaacccccwccwycvndtdgnmchwhy

SEQ ID NO: 6 - DNA, 21 bp, Artificial Sequence
  Description of Artificial Sequence Synthetic primer
gcactatattacaccaacccc

SEQ ID NO: 7 - DNA, 17 bp, Artificial Sequence
  Description of Artificial Sequence Synthetic primer
catgcawctcmcccgtm

SEQ ID NO: 8 - DNA, 34 bp, Artificial Sequence
  Description of Artificial Sequence Synthetic primer
taaccaactggcactatattacaccaaccccaat

SEQ ID NO: 9 - DNA, 38 bp, Artificial Sequence
  Description of Artificial Sequence Synthetic primer
  modified_base (5)..(5): a, c, t, or g
  modified_base (38)..(38): a, c, t, or g
vbsbnrdwwkcatgcawctcmcccgtmcattatytsbn

SEQ ID NO: 10 - DNA, 14 bp, Artificial Sequence
  Description of Artificial Sequence Synthetic probe
cgaaccccacctcc

SEQ ID NO: 11 - DNA, 34 bp, Artificial Sequence
  Description of Artificial Sequence Synthetic probe
  modified_base (26)..(26): a, c, t, or g
  modified_base (29)..(29): a, c, t, or g
caaccccaatcgaaccccwccwycvnddnmhwhy

SEQ ID NO: 12 - DNA, 17 bp, Artificial Sequence
  Description of Artificial Sequence Synthetic primer
catgcatctcccccgta

SEQ ID NO: 13 - DNA, 38 bp, Artificial Sequence
  Description of Artificial Sequence Synthetic primer
ggccagaattcatgcatctcccccgtacattatttsta

SEQ ID NO: 14 - DNA, 14 bp, Artificial Sequence
  Description of Artificial Sequence Synthetic probe
cgaaccccwccwyc

SEQ ID NO: 15 - DNA, 38 bp, Artificial Sequence
  Description of Artificial Sequence Synthetic probe
caaccccaatcgaacccccacctccccgtagaacattc

SEQ ID NO: 16 - DNA, 34 bp, Artificial Sequence
  Description of Artificial Sequence Synthetic probe
caaccccaatcgaaccccacctccccgaaaattc

SEQ ID NO: 17 - DNA, 85 bp, Artificial Sequence
  Description of Artificial Sequence Synthetic oligonucleotide
  modified_base (51)..(51): a, c, t, g, unknown or other
  modified_base (58)..(58): a, c, t, g, unknown or other
  modified_base (60)..(61): a, c, t, g, unknown or other
  modified_base (65)..(65): a, c, t, g, unknown or other
tatattrcaccaaccccaatcgaacyccmyhtyybbdtdvayhhhgbhvvntctgddndnngvgncrstaagcaaaataatgtac

SEQ ID NO: 18 - DNA, 68 bp, Artificial Sequence
  Description of Artificial Sequence Synthetic oligonucleotide
  CDS (1)..(60)
tatattacaccaaccccaatcgaaccccacctccccgaaaattccgccaaatccataaaataatgtac

SEQ ID NO: 19 - protein, 20 aa, Artificial Sequence
  Description of Artificial Sequence Synthetic peptide
YITPTPIEPHLPENSAKSIK

SEQ ID NO: 20 - DNA, 68 bp, Artificial Sequence
  Description of Artificial Sequence Synthetic oligonucleotide
tatattacaccaaccccaatcgaaccccacctcccgtgaaaatccgcaaatttgggaaaataatgtac

SEQ ID NO: 21 - DNA, 69 bp, Artificial Sequence
  Description of Artificial Sequence Synthetic oligonucleotide
tatattacaccaaccccaatcgaaccccacctccccgtgacaaccccctaaaatcccgaaataatgtac

SEQ ID NO: 22 - DNA, 69 bp, Artificial Sequence
  Description of Artificial Sequence Synthetic oligonucleotide
tatattacaccaaccccaatcgaaccccacctccccgtgacaaccccctaaaattgcgaaataatgtac

SEQ ID NO: 23 - DNA, 68 bp, Artificial Sequence
  Description of Artificial Sequence Synthetic oligonucleotide
tatattacaccaaccccaatcgaaccccacctccccgtaaaaatccaaaaatcaacgaaataatgtac

SEQ ID NO: 24 - DNA, 68 bp, Artificial Sequence
  Description of Artificial Sequence Synthetic oligonucleotide
  modified_base (52)..(68): a, c, t, g, unknown or other
tatattacaccaaccccaatcgaaccccacctccgcrtgacaacccccgaannnnnnnnnnnnnnnnn

SEQ ID NO: 25 - DNA, 68 bp, Artificial Sequence
  Description of Artificial Sequence Synthetic oligonucleotide
tatattacaccaaccccaatcgaaccccacctccccgacaaaatcgcattttccataaaataatgtac

SEQ ID NO: 26 - DNA, 68 bp, Artificial Sequence
  Description of Artificial Sequence Synthetic oligonucleotide
tatattacaccaaccccaatcgaaccccacctcccgtgaaaatctggaaaatccccaaaataatgtac

SEQ ID NO: 27 - DNA, 68 bp, Artificial Sequence
  Description of Artificial Sequence Synthetic oligonucleotide
tatattacaccaaccccaatcgaaccccacctccccgacaaaaccacaaaatccacaaaataatgtac

SEQ ID NO: 28 - DNA, 69 bp, Artificial Sequence
  Description of Artificial Sequence Synthetic oligonucleotide
tatattacaccaaccccaatcgaaccccacctccccgtgacaacccccattttcggacaaataatgtac

SEQ ID NO: 29 - DNA, 68 bp, Artificial Sequence
  Description of Artificial Sequence Synthetic oligonucleotide
tatattacaccaaccccaatcgaaccccacctccccgwsaaawycmcaaaatyggyaaaataatgtac

SEQ ID NO: 30 - DNA, 69 bp, Artificial Sequence
  Description of Artificial Sequence Synthetic oligonucleotide
tatattacaccaaccccaatcgaaccccacctccccgtaaaaaatcccaaaatcacggaaataatgtac

SEQ ID NO: 31 - DNA, 68 bp, Artificial Sequence
  Description of Artificial Sequence Synthetic oligonucleotide
tatattacaccaaccccaatcgaaccccacctccccgtaaattccaccgattttcagaaataatgtac

SEQ ID NO: 32 - DNA, 68 bp, Artificial Sequence
  Description of Artificial Sequence Synthetic oligonucleotide
tatattacaccaaccccaatcgaaccccacctccgcgtgacaacccctaaaatcccaaaataatgtac

SEQ ID NO: 33 - DNA, 69 bp, Artificial Sequence
  Description of Artificial Sequence Synthetic oligonucleotide
tatattacaccaaccccaatcgaactccacttccctgagattttcaagatttaaagtaaaataatgtac

SEQ ID NO: 34 - DNA, 69 bp, Artificial Sequence
  Description of Artificial Sequence Synthetic oligonucleotide
tatattacaccaaccccaatcgaactccacattcctgaaattttagcattttaaagcaaaataatgtac

SEQ ID NO: 35 - DNA, 69 bp, Artificial Sequence
  Description of Artificial Sequence Synthetic oligonucleotide
tatattacaccaaccccaatcgaactccacctccctgraatttccaactccgaaagcaaaataatgtac

SEQ ID NO: 36 - DNA, 69 bp, Artificial Sequence
  Description of Artificial Sequence Synthetic oligonucleotide
tatattacaccaaccccaatcgaactccacctccctgaaattttagagattagaagcaaaataatgtac

SEQ ID NO: 37 - DNA, 69 bp, Artificial Sequence
  Description of Artificial Sequence Synthetic oligonucleotide
tatattacaccaaccccaatcgaactccctctttttgaaataatgccctgagatagtaaaataatgtac

SEQ ID NO: 38 - DNA, 69 bp, Artificial Sequence
  Description of Artificial Sequence Synthetic oligonucleotide
tatattacaccaaccccaatcgaactccctctttttgaaaatagccactttaatagkaaaataatgtac

SEQ ID NO: 39 - DNA, 69 bp, Artificial Sequence
  Description of Artificial Sequence Synthetic oligonucleotide
tatattacaccaaccccaatcgaactccacatttctgaaaatatcacctctgaaagcaaaataatgtac

SEQ ID NO: 40 - DNA, 69 bp, Artificial Sequence
  Description of Artificial Sequence Synthetic oligonucleotide
tatattacaccaaccccaatcgaactccacattcctgaaaactctaggttttaaagtaaaataatgtac

SEQ ID NO: 41 - DNA, 69 bp, Artificial Sequence
  Description of Artificial Sequence Synthetic oligonucleotide
tatattacaccaaccccaatcgaactccacttccctgaaaatttccagattaatagtaaaataatgtac

SEQ ID NO: 42 - DNA, 69 bp, Artificial Sequence; identical to SEQ ID NO: 37
  Description of Artificial Sequence Synthetic oligonucleotide

(19) United States
(12) Patent Application Publication
(10) Pub. No.: US 2019/0093179 A1
(43) Pub. Date: Mar. 28, 2019

(54) OLIGONUCLEOTIDES AND USE THEREOF

(71) Applicants: Simone KANN, Veitshoechheim (DE); Jessica HANSEN, Hamburg (DE); Juergen SIEVERTSEN, Hamburg (DE)

(72) Inventors: Simone KANN, Veitshoechheim (DE); Jessica HANSEN, Hamburg (DE); Juergen SIEVERTSEN, Hamburg (DE)

(21) Appl. No.: 15/744,135

(22) PCT Filed: Jul. 12, 2016
(86) PCT No.: PCT/EP2016/066561

§ 371 Date: Jan. 12, 2018

(30) Foreign Application Priority Data
Jul. 13, 2015 (DE) 102015111267.1
Aug. 7, 2015 (DE) 102015113038.6

(51) Int. Cl. C12Q 1/6893; C07H 21/00

(52) U.S. Cl. CPC C12Q 1/6893; C07H 21/00

(57) ABSTRACT

The invention relates to oligonucleotides comprising, or consisting of, the nucleotide sequence SEQ ID NO 4 or to oligonucleotides having nucleotide sequences homologous thereto; or to oligonucleotides having a nucleotide sequence complementary to the nucleotide sequence SEQ ID NO 4 or to oligonucleotides having nucleotide sequences homologous to the complementary nucleotide sequence; or to oligonucleotides having, in view of the reading direction 5′→3′, a nucleotide sequence having an opposite reading direction to the nucleotide sequence SEQ ID NO 4, or to oligonucleotides having nucleotide sequences homologous to the opposite reading direction nucleotide sequence:

SEQ ID NO 4

cgaacccc*a cctcc 15,

wherein * represents insert “c”. The invention also relates to the use of the oligonucleotides for a real time PCR.

The present invention relates to oligonucleotides and relates to the use of said oligonucleotides for medical diagnosis. In particular, the invention relates to oligonucleotides which may be used for a real time polymerase chain reaction (RT-PCR) in the diagnosis of Chagas Disease.

Chagas Disease is a parasitic disease and is induced by the protozoal pathogen Trypanosoma cruzi (T cruzi). The pathogen is transmitted by various bloodsucking Triatomes, mainly. Documented are also congenital transmissions, transmissions by blood transfusions and organ transplantations, transmissions by contaminated food and transmissions by laboratory accidents.

More than 150 species of domestic and wild animals serve as reservoirs, but also asymptomatically infected humans contribute to a spread of the disease. 100 million living humans live with the risk of becoming infected, and about 18 million people are infected already. Hence, Chagas Disease is No. 6 of the “Global Burden Diseases” and, therefore, is a major problem for Latin and Central America. Also by migration of infected individuals to the U.S.A. and to Spain and Portugal, an increase of cases can be detected.

Various Triatome species (family Reduviidae, sub-family Triatomae) serve as vectors. They ingest the pathogen when sucking blood. The pathogens multiply in the insect and are transmitted, when the Triatome is sucking blood from a new host, e. g. a human individual, by excretion of infected faeces.

The Triatome's bite is painless in most cases, but leaves a skin lesion and an itch. As a reaction of the itching, the bitten person (mainly without intention) rubs the infected Triatome faeces into the wound.

Also possible is an infection via the mucosae of the eyes (conjunctiva), of the nose and of the mouth. Within the tissue, the pathogens invade into the host cell and multiply by bisection, thereby forming so-called “pseudocysts”. Finally, the pseudocysts burst and release the parasites, which then are transported via the bloodstream to new host cells, thereby initiating further multiplying cycles. In the course of the next “blood meal”, further Triatomes are infected, and even low pathogen concentrations in the blood are sufficient to achieve such an infection. Triiatomes are night-active animals. They may reach an age of 2 years and multiply by deposition of their eggs. Their radius of activity is about several hundred meters. All developmental stages (adults, larvae) and both sexes suck blood. Once infected, they will remain infectious for their entire life.

In a human, Chagas disease goes through several stages. In the acute stage, a local inflammation reaction (chagome) may occur, causing a swelling of adjacent lymphatic nodes. If such a swelling is close to the eyes, one finds the characteristic Romaña sign, a conjunctivitis accompanied by an eyelid edema and a preauricular swelling of the lymphatic nodes. The Romaña sign is found rarely, only (<5%). The unspecific symptoms of the disease appear like flu symptoms. Generally, the symptoms vanish spontaneously, often within 2 to 4 weeks. Only in a few cases, this stage is accompanied by a meningoencephalitis or myocarditis with letal outcome.

The above stage is followed by a latency phase showing a positive serological test and—occasionally—also a low parasitemia. This stage is also called the indeterminant or indeterminant form. Usually, this phase is free of symptoms, but medical checks revealed discrete disorders of the patients' neural system already.

30 to 40% of the affected patients develop a chronic stage going along with severe pathologies of the heart (cardiomyopathy, sudden cardiac death, etc.) or of the gastrointestinal tract (megacolon, megaesophagus, etc.).

Although Chagas Disease could be diagnosed in the acute phase, as there the parasitic load is high (by blood smear, concentration methods, buffy-coat method, etc.), the diagnosis is not made in most of the cases. Reason therefore might be that either the doctor is not consulted overall or the symptoms are not connected to Chagas Disease. Hence, in most of the cases a diagnosis is made serologically at a later time.

Presently, two compounds are available as the therapeutic agents: Benznidazol (drug products: Ragonil®, Radanil®) and Nifurtimox (drug product: Lampit®). Due to its slightly better side effect profile, Benznidazol is the compound of first choice. However, both compounds are toxic and should be administered to patients under hospital control conditions at the outset. The compounds destroy the parasites in the blood and, hence, are especially successfully when administered to patients being in the acute stage. The treatments are long term applications (Benznidazol: 60 to 90 days; Nifurtimox: 90 to 120 days). Pros and cons of the treatment must be evaluated by the attending physician carefully, in particular in cases of chronical infections. The compounds are contraindicated in pregnant women.

In order to break the cycle of infections, next to counter- and prevention measures (improvement of living conditions, fumigations, educational campaigns etc.) good diagnostic kits and methods are needed to detect and treat Chagas Disease early and to prevent a further spread by reservoirs (humans and animals). Blood and organ donors should be screened for Chagas Disease infections, and reactivations under immune suppression should be detected and treated. Therefore, novel diagnostic kits and methods are needed, having a better sensitivity and specificity than known means and tools.

Moreover, it is important for all diagnostic approaches to differentiate T. cruzi from other related (and partially apathogenic) Trypanosoma species (like T. rangeli, for example). A treatment with the above-mentioned compounds following a “positive” diagnosis result is not justifiable, if the positive diagnosis result is incorrectly based on a detection of a non-pathogenic pathogen.

By now, a “Gold Standard” for diagnosing Chagas disease is not available. Usually, in the chronic stage, serological methods are applied. Due to the low pathogen levels mainly found in chronic and chronic indeterminant forms, the PCR was already described for diagnostic use next to other methods in the state of the art.

In a large multicenter study (A G Schijman et al., (2011), International Study to Evaluate PCR Methods for Detection of Trypanosoma cruzi DNA in Blood Samples from Chagas Disease Patients, PLoS Negl Trop Dis 5 (1): e931), 48 PCR's were tested against each other. The authors concluded that 4 PCR's were declared to be the leading ones. Of those, one was the conventional Gel PCR (LbQ IDNA), and the other three were the Sat DNA PCR's LbD2-Sat-DNA-PCR, LbD3-Sat-DNA-PCR and LbF1-Sat-DNA-PCR. The last-mentioned one (LbF1-Sat-DNA PCR) is referred to as “TCZ-PCR” in the following

The approach reported by Schijman et al. was picked up by “Y Qvarnström et al., (2012), Sensitive and Specific Detection of Trypanosoma cruzi DNA in Clinical Specimens using a multi-Target Real-Time PCR Approach, PLoS Negl Trop Dis 6 (7): e1689): They compared an already known positive Chargas collective to the results of Mini-Satelite-TCZ-PCR, the leading kinetoplast PCR (kDNA-PCR) and to the 18s-rRNA-PCR which is considered as a particular specific one. From the data, the following sensitivities and specificities may be calculated:

78%, resp. 40%, for kDNA-PCR;
63%, resp. 100%, for TCZ-PCR;
6%, resp. 100%, for 18s-rRNA-PCR.

The authors concluded that a reliable diagnosis for Chagas disease could be made only for those patients for whom positive test results were achieved in all three cases. Each sample providing only one positive test result in the kDNA-PCR and/or TCZ-PCR requires a further clarification. However, any further clarification, in most local setting, remains theory: Additional diagnosis measures are not taken for cost reasons and/or due to the lack of suitable medical equipment. As a result, the diagnosis in practice often stays indefinite, and a therapy of the disease cannot be initiated, therefore.

Nevertheless, the conclusions from the above-mentioned publications result into the statement that Real Time PCR (RT-PCR) has a high priority.

Moreover, the methods used for the detection of acute or congenital infections, for the control of therapy success, for reactivating under immunosuppression or for detecting pathogen reservoirs or for screenings before organ transplantations are insufficient, still.

In the context of the present invention, a sample collective was examined which distinguished from the Qvarnström collective by a larger number volunteers (1009 vs. 119—Qvarnström, loc. cit.), the existence of all age groups and the existence of different stages of Chagas Disease (acute, chronic, chronic-indeterminant, negative).

In the context of own experiments, it could be shown in particular that the kDNA-PCR was leading to many false positive results. This was due to cross-reactions with Trypanosoma rangeli, mainly. Hence, it can be summarized that the actually most efficient PCR's (RT-PCR's) are helpful, but can just be used supportively when making the Chagas diagnose.

Without wanting to be fixed to this explanation, one of the reasons for that might be that the (RT-) PCR's used by now focus on structures in the cell which either are highly conserved (18s-rRNA-PCR) and, hence, very specific or which are existing in many copies in different parasite genomes (for example also in Leishmania spp.) (kinetoplast kDNA-PCR) and, therefore, show a false high sensitivity.

A further problem is created by low pathogen amounts as they often occur in chronic or chronic-indeterminant forms of Chagas Disease. Difficulties are also caused by the large genetic variability of the pathogens. Also these factors explain the difficulties for sensitivities and specificities.

In the context of the present invention, samples out of a high endemic area in Colombia were analyzed by rapid tests or ELISA, Immunofluorescence as well as with the real-time-PCR's (RT-PCR's) kDNA-PCR, TCZ-PCR and 18s-rRNA-PCR which—as reported above: Schijman et al.; Qvarnström et al.—belong to the leading PCR's, presently.

For a further evaluation, about 87 kDNA-PCR amplification products were cloned and sequenced.

Starting from the sequencing results, an assignment of the isolates was performed via BLAST in a NCBI to identify the underlying pathogen.

The clones for Trypanosoma cruzi were aligned against each other and against the clones of Trypanosoma rangeli as well as against sequences already available in the data base.

By doing so, a region was determined in which the sequences of all examined Trypanosoma cruzi noticeably differ from the Trypanosoma rangeli.

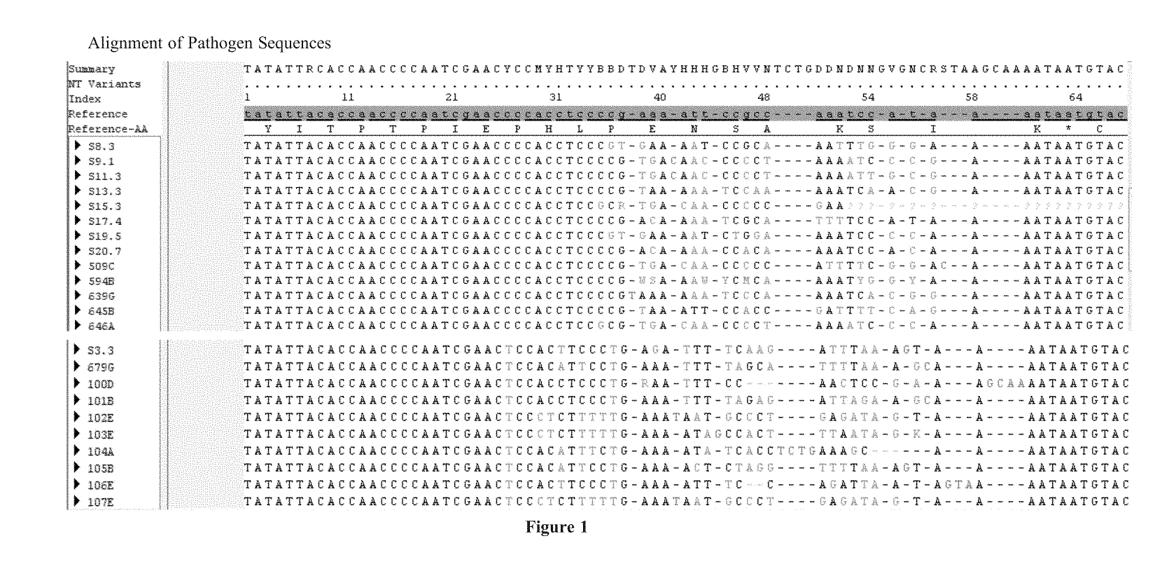

The pathogen sequence alignment is shown in FIG. 1.

Hence, the invention relates to oligonucleotides (i) having a sequence of SEQ ID NO 3 or to oligonucleotides having nucleotide sequences homologous thereto; or (ii) to oligonucleotides having a nucleotide sequence complementary to the nucleotide sequence SEQ ID NO 3 or to oligonucleotides having nucleotide sequences homologous to the complementary nucleotide 5′→3′, a nucleotide sequence having an opposite reading direction to the nucleotide sequence SEQ ID NO 3 or to oligonucleotides having nucleotide sequences homologous to the opposite reading direction nucleotide sequence:

SEQ ID NO 3

cgaacccc*w ccwyc 15,

wherein w represents a or t,
y represents c or t; and
* represents an insert “c”.

Preferred embodiments of this part of the invention are claimed in the dependent claims 2 to 7 and are described exemplarily below.

The invention further relates to oligonucleotides having the sequences SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO14, SEQ ID NO 15 and/or SEQ ID NO 16 in accordance with the subsequent detailed description for use in the amplification of pathogen nucleotide sequences, preferably for an exclusive amplification of Trypanosoma cruzi DNA sequences, in a mixture of DNA of various origin, particularly preferred by PCR, mostly preferred by real time PCR.

The invention further relates to the use of the oligonucleotides having the sequences SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO14, SEQ ID NO 15 and/or SEQ ID NO 16 in accordance with the subsequent detailed description for detecting the presence or absence of DNA of Trypanosoma cruzi in the body of an animal or of a human, preferably in the body of an insect, of poultry, of a mammal or of a human, more preferred in a sample of a body tissue or in a body liquid of poultry, of a mammal or of a human, mostly preferred in a tissue sample or in a blood sample or in a plasma sample or in a serum sample of a human.

The invention further relates to the use of the oligonucleotides having the sequences SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO14, SEQ ID NO 15 and/or SEQ ID NO 16 in accordance with the subsequent detailed description for a diagnosis of Chagas disease in the body of an animal or of a human, preferably in the body of an insect, of poultry, of a mammal or of a human, more preferred in a sample of a body tissue or in a body liquid of poultry, of a mammal or of a human, mostly preferred in a tissue sample or in a blood sample or in a plasma sample or in a serum sample of a human.

Finally, according to claim 11, the invention also relates to oligonucleotides (i) having a sequence of SEQ ID NO 1 or to oligonucleotides having nucleotide sequences homologous thereto; or (ii) to oligonucleotides having a nucleotide sequence complementary to the nucleotide sequence SEQ ID NO 1 or to oligonucleotides having nucleotide sequences homologous to the complementary nucleotide sequence; or (iii) to oligonucleotides having, in view of the reading direction 5′→3′, a nucleotide sequence having an opposite reading direction to the nucleotide sequence SEQ ID NO 1 or to oligonucleotides having nucleotide sequences homologous to the opposite reading direction nucleotide sequence:

SEQ ID NO 1

ccccwccwcc vnd 13,

wherein w represents a or t;
v represents a or c or g;
d represents a or g or t; and
n represents a or c or g or t.

Preferred embodiments of this part of the invention in the form or oligonucleotides having the SEQ ID NO 2

SEQ ID NO 2

ccccacctcc ccg 13

are claimed in dependent claim 12 and are described exemplarily below.

Hence, in the framework of the present invention, we created a nucleotide sequence of a probe specific for the distinguishing region in the Trypanosoma cruzi DNA, which probe is suitable for the use in the real time PCR, as well as forward primers and reverse primers which may be combined with said probe.

Details of the approach as conducted may be derived from the experimental data below and are explained below.

In the framework of the present invention, we claim—and describe in preferred embodiments in the description below—oligonucleotides which are characterized by SEQ ID's NO's 1 to 16 in detail mentioned below in the sequence listing belonging to the description of the invention.

The term “oligonucleotides” as referred to in the claims and in the present description means linear sequences of up to 40 nucleotides covalently bound to each other via phosphoric acid diester bridges from the 3′ carbon atom of one nucleotide to the 5′ carbon atom of the following nucleotide. These nucleotides usually are abbreviated (also in the present claims and in the description of the invention) by the small letters “a” (for adenine), “c” (for cytosine), “g” (for guanine), and “t” (for thymine). Linear sequences are represented by the covalent sequence of single nucleotides. Conventionally, the indicated nucleotide sequence is read in the direction 5′→3′.

In the frame of the present invention, the oligonucleotides of the present invention are always understood
(i) as oligonucleotides having the indicated sequence SEQ ID NO or as oligonucleotides having nucleotide sequences homologous thereto; or
(ii) as oligonucleotides having a nucleotide sequence complementary to the nucleotide sequence SEQ ID NO or as oligonucleotides having nucleotide sequences homologous to the complementary nucleotide sequence; or
(iii) as oligonucleotides having, in view of the reading direction 5′→3′, a nucleotide sequence having an opposite reading direction to the nucleotide sequence SEQ ID NO or as oligonucleotides having nucleotide sequences homologous to the opposite reading direction nucleotide sequence.

For cases of nucleotide sequences being homologous to an indicated nucleotide sequence, specific letters are used for the homologous nucleotides—also as usual—which specific letters can be derived specifically from the claims and from the specification below. Inserts occurring single cases are characterized by specifically defined symbols (*, **, §, #).

Below, the invention is described by referring to nucleotide sequences of preferred embodiments also shown in the sequence listing below. However, the invention is not restricted to the preferred embodiments described. Primarily, these preferred embodiments allow a better understanding of the invention and shall serve an exemplary illustration of the invention.

In one embodiment of the invention, oligonucleotides according to the invention have a nucleotide sequence indicated as SEQ ID NO 3:

SEQ ID NO 3

cgaacccc*w ccwyc 15,

wherein w represents a or t,
y represents c or t; and
* represents an insert “c”.

This embodiment of the invention comprises oligonucleotides having a sequence of SEQ ID NO 3 or oligonucleotides having nucleotide sequences homologous thereto; or oligonucleotides having a nucleotide sequence complementary to the nucleotide sequence SEQ ID NO 3 or oligonucleotides having nucleotide sequences homologous to the complementary nucleotide sequence; or oligonucleotides having, in view of the reading direction 5′→3′, a nucleotide sequence having an opposite reading direction to the nucleotide sequence SEQ ID NO 3 or oligonucleotides having nucleotide sequences homologous to the opposite reading direction nucleotide sequence.

In a preferred embodiment (preferred, because this embodiment provides advantageous results) which, however, does not restrict the invention, the invention relates to oligonucleotides which comprise the nucleotide sequence SEQ ID NO 14 (similar to the sequence having the SEQ ID NO 13) or to oligonucleotides having nucleotide sequences homologous thereto; or to oligonucleotides having a nucleotide sequence complementary to the nucleotide sequence SEQ ID NO 14 or to oligonucleotides having nucleotide sequences homologous to the complementary nucleotide sequence; or to oligonucleotides having, in view of the reading direction 5′→3′, a nucleotide sequence having an opposite reading direction to the nucleotide sequence SEQ ID NO 14, or to oligonucleotides having nucleotide sequences homologous to the opposite reading direction nucleotide sequence:

SEQ ID NO 14

cgaaccccwc cwyc 14,

wherein w represents a or t; and
y represents c or t.

In a different preferred embodiment (preferred, because this embodiment provides advantageous results) which, however, does not restrict the invention, the invention relates to oligonucleotides which comprise, or even consist of, the nucleotide sequence SEQ ID NO 4 (similar to the sequence having the SEQ ID NO 3 and to the sequence having the SEQ ID NO 14) or to oligonucleotides having nucleotide sequences homologous thereto; or to oligonucleotides having a nucleotide sequence complementary to the nucleotide sequence SEQ ID NO 4 or to oligonucleotides having nucleotide sequences homologous to the complementary nucleotide sequence; or to oligonucleotides having, in view of the reading direction 5′→3′, a nucleotide sequence having an opposite reading direction to the nucleotide sequence SEQ ID NO 4, or to oligonucleotides having nucleotide sequences homologous to the opposite reading direction nucleotide sequence:

SEQ ID NO 4

cgaacccc*a cctcc 15,

wherein * represents an insert “c”.

Even more preferred is an embodiment of the invention having oligonucleotides comprising, or even consist of, the nucleotide sequence SEQ ID NO 10 (similar to the sequence having the SEQ ID NO 3 and to the sequence having the SEQ ID NO 14 and to the sequence having the SEQ ID NO 4) or to oligonucleotides having nucleotide sequences homologous thereto; or to oligonucleotides having a nucleotide sequence complementary to the nucleotide sequence SEQ ID NO 10 or to oligonucleotides having nucleotide sequences homologous to the complementary nucleotide sequence; or to oligonucleotides having, in view of the reading direction 5′→3′, a nucleotide sequence having an opposite reading direction to the nucleotide sequence SEQ ID NO 10, or to oligonucleotides having nucleotide sequences homologous to the opposite reading direction nucleotide sequence:

SEQ ID NO 10

cgaaccccac ctcc 14.

In alternative embodiments, the invention relates to oligonucleotides which—as can be seen below—include nucleotide sequences of the above SEQ ID Nos 3 or 4 or 10 or 14, but have bound, to their 5′ ends and/or 3′ ends, further nucleotide sequences, or relate to oligonucleotides having nucleotide sequences homologous thereto; or relate to oligonucleotides having a nucleotide sequence complementary to the indicated nucleotide sequence or to oligonucleotides having nucleotide sequences homologous to the complementary nucleotide sequence; or relate to oligonucleotides having, in view of the reading direction 5′→3′, a nucleotide sequence having an opposite reading direction to the indicated nucleotide sequence, or to oligonucleotides having nucleotide sequences homologous to the opposite reading direction nucleotide sequence.

These oligonucleotides comprise the nucleotide sequences of the SEQ ID NO 5:

SEQ ID NO 5

caaccccaat cgaacccc*w ccwyc vnd**d §nm#hwhy 38

wherein w represents a or t;
m represents a or c;
y represents c or t;
v represents a or c or g;
d represents a or g or t;
h represents a or c or t;
n represents a or c or g or t;
* represents insert “c”;
** represents insert “t”;
§ represents insert “g”; and
# represents insert “c”.

Preferred oligonucleotides belonging to the above-mentioned group comprise oligonucleotides of the SEQ ID NO 15 or comprise oligonucleotides having nucleotide sequences homologous thereto; or comprise oligonucleotides having a nucleotide sequence complementary to the indicated nucleotide sequence or oligonucleotides having nucleotide sequences homologous to the complementary nucleotide sequence; or comprise oligonucleotides having, in view of the reading direction 5′→3′, a nucleotide sequence having an opposite reading direction to the indicated nucleotide sequence or oligonucleotides having nucleotide sequences homologous to the opposite reading direction nucleotide sequence having nucleotide sequences of SEQ ID NO 15:

SEQ ID NO 15

caaccccaat cgaacccc*a cctccccg**a §aa#attc 38

wherein * represents insert “c”;
** represents insert “t”;
§ represents insert “g”; and
# represents insert “c”.

Also preferred oligonucleotides of the above-mentioned group comprise oligonucleotides of the SEQ ID NO 11 or comprise oligonucleotides having nucleotide sequences homologous thereto; or comprise oligonucleotides having a nucleotide sequence complementary to the indicated nucleotide sequence or oligonucleotides having nucleotide sequences homologous to the complementary nucleotide sequence; or comprise oligonucleotides having, in view of the reading direction 5′→3′, a nucleotide sequence having an opposite reading direction to the indicated nucleotide sequence or oligonucleotides having nucleotide sequences homologous to the opposite reading direction nucleotide sequence having nucleotide sequences of SEQ ID NO 11:

SEQ ID NO 11

caaccccaat cgaaccccwc cwycvnddnm hwhy 34,

wherein w represents a or t;
m represents a or c;
y represents c or t;
v represents a or c or g;
d represents a or g or t;
h represents a or c or t; and
n represents a or c or g or t.

Also preferred oligonucleotides of the above-mentioned group comprise oligonucleotides or comprise oligonucleotides having nucleotide sequences homologous thereto; or comprise oligonucleotides having a nucleotide sequence complementary to the indicated nucleotide sequence or oligonucleotides having nucleotide sequences homologous to the complementary nucleotide sequence; or comprise oligonucleotides having, in view of the reading direction 5′→3′, a nucleotide sequence having an opposite reading direction to the indicated nucleotide sequence or oligonucleotides having nucleotide sequences homologous to the opposite reading direction nucleotide sequence having nucleotide sequences of SEQ ID NO 16:

SEQ ID NO 16

caaccccaat cgaaccccac ctccccgaaa attc 34.

The last-mentioned group of oligonucleotides having SEQ ID NO 16 is particularly preferred in view of the achieved detection of Trypanosoma cruzi pathogen sequences.

In further preferred embodiments of the invention which may be realized alone or together with further features of the invention and are intended not to restrict the invention, the invention relates to oligonucleotides of the above-defined SEQ ID NO 3 or SEQ ID NO 4 or SEQ ID NO 5 or SEQ ID NO 10 or SEQ ID NO 11 or SEQ ID NO 14 or SEQ ID NO 15 or SEQ ID NO 16 having one or several marking(s) at one or at both of their 5′ end(s) and/or 3′ end(s). Such marking(s) which may serve specific purposes in the subsequent use of the oligonucleotides are known to a skilled person generally and, hence, may be selected in accordance with the circumstances of a single case from markings known from the prior art. In further preferred embodiments, one of the markings is, or several of the markings are, marker(s), even more preferred fluorescence marker(s) or quencher(s). Here, known fluorescence markers or quenchers as FAM or BHQ1 may be mentioned exemplarily.

For example (and without any restriction), oligonucleotides having the SEQ ID Nos 3, 4, 5, 10, 11, 14, 15 and/or 16 in accordance with the above description may be used as probes for the PCR; in accordance with the invention, they may serve particularly preferred as probes for Real Time PCR, but a use of the oligonucleotides of the invention is not restricted to a use as mentioned above.

In further preferred embodiments of the invention which may be realized alone or together with further features of the invention and which are intended not to restrict the invention, the afore-described oligonucleotides of the SEQ ID NO 3 or SEQ ID NO 4 or SEQ ID NO 5 or SEQ ID NO 10 or SEQ ID NO 11 or SEQ ID NO 14 or SEQ ID NO 15 or SEQ ID NO 16 according to the above detailed exemplary description are found in combination with one or more further oligonucleotide(s) selected from the group consisting of forward primers and reverse primers.

It is even more preferred that, for example, oligonucleotides of the SEQ ID NO 3 or SEQ ID NO 4 or SEQ ID NO 5 or SEQ ID NO 10 or SEQ ID NO 11 or SEQ ID NO 14 or SEQ ID NO 15 or SEQ ID NO 16 can be combined with a primer (1), for example in the form of oligonucleotides having a nucleotide sequence of SEQ ID NO 6, or in the form of oligonucleotides having nucleotide sequences homologous thereto; or in the form of oligonucleotides having a nucleotide sequence complementary to the nucleotide sequence SEQ ID NO 6 or in the form of oligonucleotides having nucleotide sequences homologous to the complementary nucleotide sequence; or in the form of oligonucleotides having, in view of the reading direction 5′→3′, a nucleotide sequence having an opposite reading direction to the nucleotide sequence SEQ ID NO 6 or in the form of oligonucleotides having nucleotide sequences homologous to the opposite reading direction nucleotide sequence:

SEQ ID NO 6

gcactatatt acaccaaccc c 21

and/or can be combined with a primer (2), for example in the form of oligonucleotides having a nucleotide sequence of SEQ ID NO 7, or in the form of oligonucleotides having nucleotide sequences homologous thereto; or in the form of oligonucleotides having a nucleotide sequence complementary to the nucleotide sequence SEQ ID NO 7 or in the form of oligonucleotides having nucleotide sequences homologous to the complementary nucleotide sequence; or in the form of oligonucleotides having, in view of the reading direction 5′→3′, a nucleotide sequence having an opposite reading direction to the nucleotide sequence SEQ ID NO 7 or in the form of oligonucleotides having nucleotide sequences homologous to the opposite reading direction nucleotide sequence:

SEQ ID NO 7

catgcawctc mcccgtm 17,

wherein w represents a or t; and
m represents a or c.

In a preferred embodiment (being a good detection basis) of oligonucleotides having the above nucleotide sequence of SEQ ID NO 7, which may be realized alone or together with other features of the invention and which is intended not to restrict the invention, or of oligonucleotides having nucleotide sequences homologous thereto; or of oligonucleotides having a nucleotide sequence complementary to the nucleotide sequence SEQ ID NO 7 or of oligonucleotides having nucleotide sequences homologous to the complementary nucleotide sequence; or of oligonucleotides having, in view of the reading direction 5′→3′, a nucleotide sequence having an opposite reading direction to the nucleotide sequence SEQ ID NO 7 or of oligonucleotides having nucleotide sequences homologous to the opposite reading direction nucleotide sequence, the nucleotide sequence is one of SEQ ID NO 12:

SEQ ID NO 12

catgcatctc ccccgta 17.

Preferably, the primer (1) is a forward primer and/or the primer (2) is a reverse primer. In another embodiment of the invention, the primer (2) is a forward primer and/or the primer (1) is a reverse primer.

As the primers (1) and/or (2), oligonucleotides may also be employed which contain further nucleotide sequences bound to their 5′ end and/or to their 3′ end and contain in each case one oligonucleotide sequence in accordance with the above SEQ ID Nos 6 and/or 7 (or at least a homologous thereof):

Hence, the invention also relates to oligonucleotides of the above-defined SEQ ID Nos 4, 5, 6, 7, 10, 11 and 12, which comprise:

as the primer (1), nucleotide sequences of SEQ ID NO 8 or oligonucleotides having nucleotide sequences homologous thereto; or oligonucleotides having a nucleotide sequence complementary to the nucleotide sequence SEQ ID NO 8 or oligonucleotides having nucleotide sequences homologous to the complementary nucleotide sequence; or oligonucleotides having, in view of the reading direction 5′→3′, a nucleotide sequence having an opposite reading direction to the nucleotide sequence SEQ ID NO 8 or oligonucleotides having nucleotide sequences homologous to the opposite reading direction nucleotide sequence:

SEQ ID NO 8

taaccaactg gcactatatt acaccaaccc caat 34,

and/or which comprise, as the primer (2), nucleotide sequences of SEQ ID NO 9 or oligonucleotides having nucleotide sequences homologous thereto; or oligonucleotides having a nucleotide sequence complementary to the nucleotide sequence SEQ ID NO 9 or oligonucleotides having nucleotide sequences homologous to the complementary nucleotide sequence; or oligonucleotides having, in view of the reading direction 5′→3′, a nucleotide sequence having an opposite reading direction to the nucleotide sequence SEQ ID NO 9 or oligonucleotides having nucleotide sequences homologous to the opposite reading direction nucleotide sequence:

SEQ ID NO 9

vbsbnrdwwk catgcawctc mcccgtmcat tatyt*bn 38

wherein s represents c or g;
w represents a or t;
r represents a or g;
y represents c or t;
k represents g or t;
v represents a or c or g;
d represents a or g or t;
b represents c or g or t;
n represents a or c or g or t; and
* represents insert “s”.

In a preferred embodiment (because of being a good detection basis) of oligonucleotides having the above nucleotide sequence of SEQ ID NO 9, which may be realized alone or together with other features of the invention and which is intended not to restrict the invention, or of oligonucleotides having nucleotide sequences homologous thereto; or of oligonucleotides having a nucleotide sequence complementary to the nucleotide sequence SEQ ID NO 9 or of oligonucleotides having nucleotide sequences homologous to the complementary nucleotide sequence; or of oligonucleotides having, in view of the reading direction 5′→3′, a nucleotide sequence having an opposite reading direction to the nucleotide sequence SEQ ID NO 9 or of oligonucleotides having nucleotide sequences homologous to the opposite reading direction nucleotide sequence, the nucleotide sequence is one of SEQ ID NO 13:

SEQ ID NO 13

ggccagaatt catgcatctc ccccgtacat tattt*ta 38

wherein * represents insert “5”.

Also in this case, it is further preferred that the primer (1) is a forward primer and/or the primer (2) is a reverse primer, or the primer (2) is a forward primer and/or the primer (1) is a reverse primer.

Furthermore the invention, in a further aspect, relates to oligonucleotides of the SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15 and SEQ ID NO 16 as described above in detail for the use in the amplification of pathogen nucleotide sequences. In particular, the oligonucleotides in accordance with the above-defined sequences of SEQ ID Nos 3 to 16 have proven to be successful in an exclusive amplification of Trypanosoma cruzi DNA sequences in a mixture of DNA's of various origin, particularly preferred by PCR, mostly preferred in accordance with the invention by real time PCR.

The results of conducting real time PCR including the combinations of primers (forward primers and reverse primers) according to the invention with an oligonucleotide probe (optionally together with marker(s)) according to the invention suggest (without wanting to be fixed to this interpretation) that the identified region of the Trypanosoma cruzi DNA in fact is a significant distinguishing feature to the DNA of Trypanosoma rangeli. Cross reactions to Trypanosoma rangeli are not observed.

Trypanosoma cruzi strains tested in the frame of the present invention are the strains Trypanosoma cruzi CL Brenner (Hg 39), Trypanosoma cruzi Typ Y (Vero) and Trypanosoma cruzi Brazil (HG 39). These strains were subjected to a detection in the new real time PCR's by using the oligonucleotides according to the present invention. On the other hand, cross reactions with Malaria tertiana and Malaria tropica and Leishmania brasiliensis could not be observed.

Hence, the invention also relates to the use of the oligonucleotides of the defined SEQ ID NO's 3 to 16 for a detection of the presence, or absence, of Trypanosoma cruzi to DNA in the body of an animal or of a human, preferably in the body of an insect, of a poultry, of a mammal or of a human, further preferred in a sample of a body tissue or of a body liquid of a poultry, of a mammal or of a human, mostly preferred in a sample of tissue or in a sample of blood or in a sample of plasma or in a sample of serum of a human.

The invention further relates the use of the oligonucleotides of the defined SEQ ID NO's 3 to 16 for a diagnosis of Chagas disease in the body of an animal or of a human, preferably in the body of an insect, of a poultry, of a mammal or of a human, further preferred in a sample of a body tissue or of a body liquid of a poultry, of a mammal or of a human, mostly preferred in a sample of tissue or in a sample of blood or in a sample of plasma or in a sample of serum of a human.

As described above, the invention also relates to oligonucleotides
(i) having the sequence SEQ ID NO 1 or to oligonucleotides having nucleotide sequences homologous thereto; or to
(ii) oligonucleotides having a nucleotide sequence complementary to the nucleotide sequence SEQ ID NO 1, or to oligonucleotides having nucleotide sequences homologous to the complementary nucleotide sequence; or to
(iii) oligonucleotides having, in view of the reading direction 5′→3′, a nucleotide sequence having an opposite reading direction to the nucleotide sequence SEQ ID NO 1, or to oligonucleotides having nucleotide sequences homologous to the opposite reading direction nucleotide sequence:

SEQ ID NO 1

ccccwccwcc vnd 13,

wherein w represents a or t;
v represents a or c or g;
d represents a or g or t; and
n represents a or c or g or t,
   which oligonucleotides are suitable for distinguishing the pathogen sequence of Trypanosoma cruzi from sequences of other Trypanosoma species, for example of Trypanosoma rangeli.

In a preferred embodiment (resulting into a good detection basis), the invention relates to oligonucleotides comprising, or even consisting of, oligonucleotides having the nucleotide sequence of SEQ ID NO 2, or to oligonucleotides having nucleotide sequences homologous thereto; or to oligonucleotides having a nucleotide sequence complementary to the nucleotide sequence SEQ ID NO 2 or oligonucleotides having nucleotide sequences homologous to the complementary nucleotide sequence; or to oligonucleotides having, in view of the reading direction 5′→3′, a nucleotide sequence having an opposite reading direction to the nucleotide sequence SEQ ID NO 2 or to oligonucleotides having nucleotide sequences homologous to the opposite reading direction nucleotide sequence:

SEQ ID NO 2

ccccacctcc ccg 13,

allowing a particularly reliable distinction of the pathogen sequence of Trypanosoma cruzi from that one of Trypanosoma rangeli, in contrast to the PCR's of the prior art described in the introduction to this invention.

### Experimental Part: Part 1:

In the PCR run presented by the following combined Table, various DNA samples of a number of volunteers were tested. All samples were examined by serology (rapid test [“Chagas Ab Rapid” obtained from Standard Diagnostics] or ELISA [Elisa in House, obtained from Laboratorio de Salud], Immunofluorescence [Immunofluorescence in House, obtained from Bernhard-Nocht-Institute for Tropical Medicine, Hamburg, Germany], kDNA-PCR, TCZ-PCR, 18s-rRNA-PCR.

Samples in the Table marked with “Cruzi”, “Rangeli” and “Homo s” were sequenced additionally.

“Neg. Probe” 1 to 4 are samples of volunteers which were tested to be “negative” in all serological tests and “negative” in all PCR tests.

“Tulahuen” indicates two positive controls in two different concentrations.

For the PCR, the following preferred primers were used:

(SEQ ID NO 6)

| | |
|---|---|
| Primer 1 (Forward): gcactatattc | 21 nucleotides |
| acaccaaccc | |

(SEQ ID NO 12)

| | |
|---|---|
| Primer 2 (Reverse): catgcatctc | 17 nucleotides |
| ccccgta | |

(SEQ ID NO 10)

| | |
|---|---|
| Probe: cgaaccccac ctcc | 14 nucleotides |

For the PCR, the following optimized reaction mix was used:

10 μl 2×Master Mix (Hot Star Taq Mastermix, obtained from Qiagen)

1 μl Primer Forward 8 pmol (SEQ ID NO 6)

1 μl Primer Reverse 8 pmol (SEQ ID NO 12)

1 μl Probe 2 pmol (SEQ ID NO 10)

2 μl MgCl₂ 25 mMol

3 μl Aqua dest..

To the above 18 μl mixture, 2 μl of each of the extracted DNA of the samples to be tested were added.

DNA of Trypanosoma tulahuen in the same concentration was used as the positive control.

The program of the cycler (Rotorgene, obtained from Qiagen) was programmed in the following way:

15 min at 95° C.; followed by

45 cycles for 15 sec at 95° C. and 60 sec at 60° C.; followed by

30 sec at 40° C.

The results can be seen from the following Tables:

TABLE 1
Experiment Information

| Run Name | Lauf definierte Proben |
|---|---|
| Run Start | 5/5/2015 3:31:52 AM |
| Run Finish | 5/5/2015 5:38:03 AM |
| Operator | PCR |
| Notes | |
| Run On Software Version | Rotor-Gene 2.1.0.9 |
| Run Signature | The Run Signature is valid. |
| Gain Green | 8. |
| Gain Yellow | 10. |
| Gain Orange | 10. |
| Gain Red | 10. |

TABLE 2
Quantitation Information

| | |
|---|---|
| Threshold | 0.00483 |
| Left Threshold | 1.000 |
| Standard Curve imported | No |
| Standard Curve (1) | N/A |
| Standard Curve (2) | N/A |
| Start normalising from cycle | 1 |
| Noise Slope Correction | Yes |
| No Template Control Threshold | %3 |
| Reaction Efficiency Threshold | Disabled |
| Normalisation Method | Dynamic Tube Normalisation |
| Digital Filter | Light |
| Sample Page | Page 1 |
| Imported Analysis Settings | |

Results:

TABLE 3

| | | | | | | | |
|---|---|---|---|---|---|---|---|
| | | | | | Ct | Given Conc | Calc Conc |
| No. | Colour | Name | Type | Ct | Comment | (Copies) | (Copies) |

| | | | | | | |
|---|---|---|---|---|---|---|
| | 1 | ▪ | Cruzi 1 | Unknown | 33.93 | |
| | 2 | □ | Cruzi 1 | Unknown | 28.89 | |
| | 3 | ▪ | Cruzi 1 | Unknown | 39.95 | |
| | 4 | ▪ | Cruzi 2 | Unknown | 32.92 | |
| | 5 | □ | Cruzi 2 | Unknown | 33.70 | |
| | 6 | ▪ | Cruzi 2 | Unknown | 35.03 | |
| | 7 | ▪ | Cruzi 3 | Unknown | 29.76 | |
| | 8 | □ | Cruzi 3 | Unknown | 30.03 | |
| | 9 | ▪ | Cruzi 3 | Unknown | 29.83 | |
| | 10 | ▪ | Cruzi 4 | Unknown | 35.73 | |
| | 11 | ▪ | Cruzi 4 | Unknown | 33.02 | |
| | 12 | ▪ | Cruzi 4 | Unknown | 36.50 | |
| ✓ | 13 | [symbol] | Cruzi 5 | Unknown | 36.38 | |
| ✓ | 14 | [symbol] | Cruzi 5 | Unknown | 35.39 | |
| ✓ | 15 | [symbol] | Cruzi 5 | Unknown | 34.02 | |
| | 16 | ▪ | Rangeli 1 | Unknown | | NEG (NTC) |
| | 17 | ▪ | Rangeli 1 | Unknown | | NEG (NTC) |
| | 18 | ▪ | Rangeli 1 | Unknown | | NEG (NTC) |
| | 19 | ▪ | Rangeli 2 | Unknown | | NEG (NTC) |
| | 20 | ▪ | Rangeli 2 | Unknown | | NEG (NTC) |
| | 21 | ▪ | Rangeli 2 | Unknown | | NEG (NTC) |
| | 22 | ▪ | Rangeli 3 | Unknown | | NEG (NTC) |
| | 23 | ▪ | Rangeli 3 | Unknown | | NEG (NTC) |
| | 24 | ▪ | Rangeli 3 | Unknown | | NEG (NTC) |
| | 25 | ▪ | Rangeli 4 | Unknown | | NEG (NTC) |
| | 26 | ▪ | Rangeli 4 | Unknown | | NEG (NTC) |
| | 27 | ▪ | Rangeli 4 | Unknown | | NEG (NTC) |
| | 28 | | Homo S. 1 | Unknown | | NEG (NTC) |
| ✓ | 29 | [symbol] | Homo S. 1 | Unknown | | NEG (NTC) |
| | 30 | ▪ | Homo S. 1 | Unknown | | NEG (NTC) |
| | 31 | ▪ | Homo S. 2 | Unknown | | NEG (NTC) |
| | 32 | ▪ | Homo S. 2 | Unknown | | NEG (NTC) |
| | 33 | ▪ | Homo S. 2 | Unknown | 37.10 | |
| ✓ | 34 | [symbol] | Homo S. 3 | Unknown | | NEG (NTC) |
| | 35 | ▪ | Homo S. 3 | Unknown | | NEG (NTC) |
| | 36 | ▪ | Homo S. 3 | Unknown | | NEG (NTC) |
| | 37 | ▪ | Homo S. 4 | Unknown | | NEG (NTC) |
| | 38 | ▪ | Homo S. 4 | Unknown | | NEG (NTC) |
| | 39 | ▪ | Homo S. 4 | Unknown | | NEG (NTC) |
| | 40 | ▪ | Neg. Probe 1 | Unknown | | NEG (NTC) |
| | 41 | ▪ | Neg. Probe 1 | Unknown | | NEG (NTC) |
| | 42 | ▪ | Neg. Probe 1 | Unknown | | NEG (NTC) |
| | 43 | ▪ | Neg. Probe 2 | Unknown | | NEG (NTC) |
| | 44 | ▪ | Neg. Probe 2 | Unknown | | NEG (NTC) |
| | 45 | ▪ | Neg. Probe 2 | Unknown | | NEG (NTC) |
| | 46 | ▪ | Neg. Probe 3 | Unknown | | NEG (NTC) |
| | 47 | ▪ | Neg. Probe 3 | Unknown | | NEG (NTC) |
| | 48 | ▪ | Neg. Probe 3 | Unknown | | NEG (NTC) |
| | 49 | ▪ | Neg. Probe 4 | Unknown | | NEG (NTC) |
| | 50 | ▪ | Neg. Probe 4 | Unknown | | NEG (NTC) |
| | 51 | ▪ | Neg. Probe 4 | Unknown | 35.17 | |
| | 52 | ▪ | Negativ - H2O | Unknown | | NEG (NTC) |
| | 53 | ▪ | Negativ - H20 | Unknown | | NEG (NTC) |
| | 54 | ▪ | Positiv Tulahuen 1:100 | Unknown | 23.09 | |
| | 55 | ▪ | Positiv Tuahuen 1:1000 | Unknown | 27.38 | |

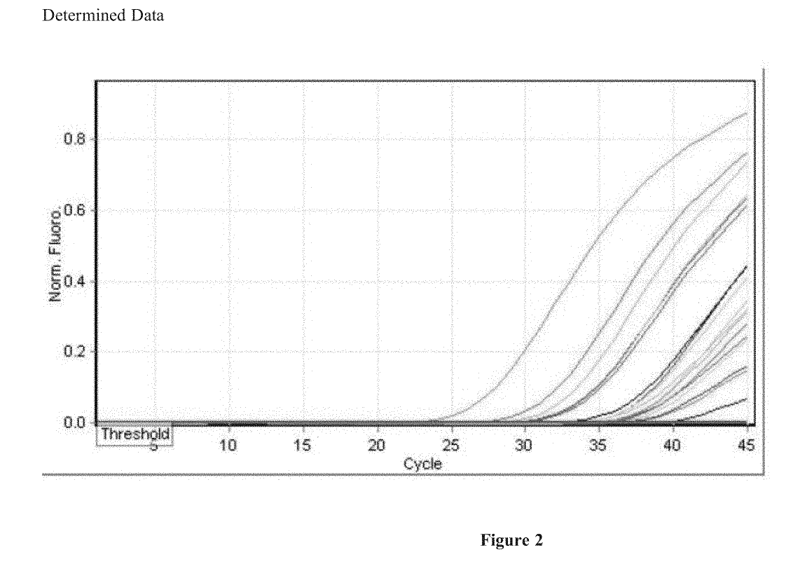

The obtained values are compared to each other graphically in FIG. 2.

As the above data together with the graph of FIG. 2 show: In all cases where the pathogen sequencing confirmed Trypanosoma cruzi to be the pathogen, the CT's were in the expected range, demonstrating a distinct detection.

In contrast, all other (related) pathogen sequences of Trypanosoma rangeli were negative in the test.

### Experimental Part: Part 2:

### Sensitivity, Specificity

The laboratory-based diagnosis of Chagas Disease is a big challenge as there is no Gold Standard available. Moreover, the results of a Chagas diagnosis depend on the stage of the disease. For chronic forms, good serologic tests are available (rapid tests, Elisa, Immunofluorescence etc.), but acute, indeterminant or congenital forms as well as relapses or therapy monitoring cannot be covered. In contrast to the indirect serological detection methods, a direct detection of the pathogen is recommended, for example by RT-PCR.

Many approaches were tried and evaluated (see the publications of Schijman et al.; of Qvarnström et al.; and others). As examples for many PCR's, Qvarnström et al. presented sensitivities and specificities of the three leading PCR methods:

| | kDNA PCR | 18s-rRNA PCR | TCZ PCR |
|---|---|---|---|
| Sensitivity | 78% | 6% | 63% |
| Specificity | 40% | 100% | 100% |

(modified in accordance with Qvarnström et al, 2012)

In this connection, the study collective tested (119) is remarkable, which mainly consisted of adults with known Chagas Disease, reactivated under immune suppression (after organ transplantations, AIDS) or congenitally-acquired infections or infections acquired after laboratory accidents. High parasitemia values could be expected for many patients of this collective. Nevertheless, the authors concluded that with these PCR's, a diagnostic support is possible, but it cannot be used for giving a reliable diagnose, except the samples are positive in all three cases of PCR's at the same time.

In the context of a study in Colombia, we became aware of the problem of Chagas Disease. We tested—in total—1009 samples for Chagas Disease collected from a highly endemic area.

From this collective consisting of all ages, including healthy persons and persons suffering from acute, chronic and chronic-indeterminant Chagas Disease forms, about 100 PCR-positive samples were sequenced. Since all samples tested by TCZ PCR and 18s rRNA PCR were also positive in the kDNA PCR test, the amplificate of kDNA PCR was cloned and sequenced. A cloning and sequencing was successful in 87 samples. Verified by sequencing, we found 65 cases for Trypanosoma cruzi, 14 for Trypanosoma rangeli and 8 for Homo sapiens.

PCR amplificates taken from six samples of the newly developed PCR were sequenced, too. All of them showed a positive proof of Trypanosoma cruzi. One of these positively sequenced samples was positive only in the newly developed PCR and was not detected by any of the other three PRC's.

Based on these results, we calculated sensitivities and specificities for the sequenced collective on behalf of all PCR's to equal conditions; the results are shown in the following Table:

| | | | | Newly |
|---|---|---|---|---|
| | kDNA PCR | 18s-rRNA PCR | TCZ PCR | developed PCR |
| Sensitivity | 89.2% | 1.5% | 20.5% | 92.3% |
| Specificity | 22.7% | 100% | 100% | 100% |

The good specificity values of the 18 s rRNA PCR and TCZ PCR could—as also described by Qvarnström et al.—be confirmed here, too. However, sensitivities were lower (many false negative results). The reason therefore could be the different collectives.

In cases of reactivation, for example, parasitemias are usually high. However, in a cross-secrional population with mainly chronic/chronic indeterminant courses, the number of parasites is usually low.

The kDNA PCR yields a good sensitivity, but has a very low specificity. The reasons for that are many false positive results in the tests, mainly caused by cross reactions with Trypanosoma rangeli. Trypanosoma rangeli is a close relative to Trypanosoma cruzi, but is considered to be apathogenic in contrast to Trypanosoma cruzi. As the only PCR test, the newly developed PCR shows both, good sensitivities and good specificities. In the tested group, no sample was classified as false positive, and all samples supposing to be negative were correctly detected to be negative.

### Inter and Intra Assay Data

For an inter assay, the positive control Tulahuen was used in a dilution of 1:100 and was tested on three different days (22 to 24 June 2015) on 10 samples per run. The variation coefficient value was 1.86%.

For an intra assay, 20 samples of the positive control Tulahuen were diluted 1:100 and 1:10.000 and were tested in one run. The variation coefficient value was 1.7% for the 1:100 dilution and was 2.4% for the 1:10,000 dilution.

### Primer Dimer Run

In a primer dimer run (also called “water run”), only primer, probe, magnesium chloride, buffer and water were mixed in order to learn about interactions of the kit components with each other. Such interactions could not be observed in any of the 30 samples tested.

### Defined Plasmid for Quantification

By means of a defined plasmid, a standard curve for a determination of the parasitemia quantity was created. For this purpose, an already known positive sample was amplified, cloned and sequenced. Simultaneously, the plasmid concentration was measured. After another confirmation of the sequence to be T. cruzi, and a measurement result of 10¹¹ plasmids/μl in the sample, the standard curve could be generated by means of a dilution series in a triple batch. The measurement of the dilution series as well as the generation of the standard curve was made separately for both, the kDNA PCR (modified in accordance with Qvarnström et al.) and also for the newly developed PCR.

This was followed by a measurement test of two known positive samples in logarithmic dilution stages for both above-mentioned PCR's.

As the detection limit, the Schijman et al. (2011) publication describes 5×10⁻¹ parasites/ml. Our studies revealed a detection limit for the kDNA PCR (modified in accordance with Qvarnström et al.) of 3×10⁻² parasites/ml.

The newly developed PCR has a detection limit of 3×10⁻⁴ parasites/ml.

As a result, the newly developed PCR can detect even very low parasitemias. This gives the possibility to improve the diagnostics in acute, congenital and chronic- indeterminant infections as well as in therapy success control, reactivations under immunosuppression, screening before organ transplantions or blood donations and disease countermeasures (for example vector/reservoir control).

### Sequence Listing

| | | |
|---|---|---|
| <110> | Name of the Applicant | Dr. Simone Kann |
| | | Steinwiese 26, 53721 |
| | | Siegburg |
| <120> | Title of the invention | Oligonucleotides |
| | | and the use thereof |
| <160> | Number of SEQ ID NOs | 16 |
| <210> | SEQ ID NO | 1 |
| <211> | Length | 13 |
| <212> | Type | — |
| <213> | Organism | Artificial sequence |
| <400> | Sequence | ccccwccwcc vnd |
| <210> | SEQ ID NO | 2 |
| <211> | Length | 13 |
| <212> | Type | — |
| <213> | Organism | Artificial sequence |
| <400> | Sequence | ccccacctcc ccg |
| <210> | SEQ ID NO | 3 |
| <211> | Length | 15 |
| <212> | Type | Probe |
| <213> | Organism | Artificial sequence |
| <400> | Sequence | cgaacccc*w ccwyc |
| <210> | SEQ ID NO | 4 |
| <211> | Length | 15 |
| <212> | Type | Probe |
| <213> | Organism | Artificial sequence |
| <400> | Sequence | cgaacccc*a cctcc |
| <210> | SEQ ID NO | 5 |
| <211> | Length | 38 |
| <212> | Type | Probe |
| <213> | Organism | Artificial sequence |
| <400> | Sequence | caaccccaat cgaacccc*w |
| | | ccwycvnd**d §nm#hwhy |
| <210> | SEQ ID NO | 6 |
| <211> | Length | 21 |
| <212> | Type | Primer |
| <213> | Organism | Artificial sequence |
| <400> | Sequence | gcactatatt |
| | | acaccaaccc c |
| <210> | SEQ ID NO | 7 |
| <211> | Length | 17 |
| <212> | Type | Primer |
| <213> | Organism | Artificial sequence |
| <400> | Sequence | catgcawctc mcccgtm |
| <210> | SEQ ID NO | 8 |
| <211> | Length | 34 |
| <212> | Type | Primer |
| <213> | Organism | Artificial sequence |
| <400> | Sequence | taaccaactg gcactatatt |
| | | acaccaaccc caat |
| <210> | SEQ ID NO | 9 |
| <211> | Length | 38 |
| <212> | Type | Primer |
| <213> | Organism | Artificial sequence |
| <400> | Sequence | vbsbnrdwwk catgcawctc |
| | | mcccgtmcat tatyt*bn |
| <210> | SEQ ID NO | 10 |
| <211> | Length | 14 |
| <212> | Type | Probe |
| <213> | Organism | Artificial sequence |
| <400> | Sequence | cgaaccccac ctcc |
| <210> | SEQ ID NO | 11 |
| <211> | Length | 34 |
| <212> | Type | Probe |
| <213> | Organism | Artificial sequence |
| <400> | Sequence | caaccccaat cgaaccccwc |
| | | cwycvnddnm hwhy |
| <210> | SEQ ID NO | 12 |
| <211> | Length | 17 |
| <212> | Type | Primer |
| <213> | Organism | Artificial sequence |
| <400> | Sequence | catgcatctc ccccgta |
| <210> | SEQ ID NO | 13 |
| <211> | Length | 38 |
| <212> | Type | Primer |
| <213> | Organism | Artificial sequence |
| <400> | Sequence | ggccagaatt catgcatctc |
| | | ccccgtacat tattt*ta |
| <210> | SEQ ID NO | 14 |
| <211> | Length | 14 |
| <212> | Type | Probe |
| <213> | Organism | Artificial sequence |
| <400> | Sequence | cgaaccccwc cwyc |
| <210> | SEQ ID NO | 15 |
| <211> | Length | 38 |
| <212> | Type | Probe |
| <213> | Organism | Artificial sequence |
| <400> | Sequence | caaccccaat cgaacccc*a |
| | | cctccccg**a §aa#attc |
| <210> | SEQ ID NO | 16 |
| <211> | Length | 34 |
| <212> | Type | Probe |
| <213> | Organism | Artificial sequence |
| <400> | Sequence | caaccccaat cgaaccccac |
| | | ctccccgaaa attc |

1.-12. (canceled)

13. An oligonucleotide
(i) having sequence SEQ ID NO 3, or an oligonucleotide having a nucleotide sequence homologous thereto; or
(ii) an oligonucleotide having a nucleotide sequence complementary to nucleotide sequence SEQ ID NO 3 or an oligonucleotide having a nucleotide sequence homologous to the complementary nucleotide sequence; or
(iii) an oligonucleotide having, in in reading direction 5′→3′, a nucleotide sequence having an opposite reading direction to nucleotide sequence SEQ ID NO 3 or an oligonucleotide having a nucleotide sequence homologous to the opposite reading direction nucleotide sequence:
SEQ ID NO 3
cgaacccc*w ccwyc 15,
wherein w represents a or t,
y represents c or t; and
* represents insert “c”.

14. The oligonucleotide of claim 13, comprising, or consisting of, nucleotide sequence SEQ ID NO 14 or an oligonucleotide having a nucleotide sequence homologous thereto; or an oligonucleotide having a nucleotide sequence complementary to nucleotide sequence SEQ ID NO 14 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 14, or an oligonucleotide having a nucleotide sequence homologous to the opposite reading direction nucleotide sequence:
SEQ ID NO 14
cgaaccccwc cwyc 14,
wherein w represents a or t; and
y represents c or t; or
comprising, or consisting of, nucleotide sequence SEQ ID NO 4 or an oligonucleotide having a nucleotide sequence homologous thereto; or an oligonucleotide having a nucleotide sequence complementary to nucleotide sequence SEQ ID NO 4 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 4, or an oligonucleotide having an nucleotide sequence homologous to the opposite reading direction nucleotide sequence:
SEQ ID NO 4
cgaacccc*a cctcc 15,
wherein * represents insert “c”.

15. The oligonucleotide of claim 14, comprising, or consisting of, nucleotide sequence SEQ ID NO 10 or an oligonucleotide having a nucleotide sequence homologous thereto; or an oligonucleotide having a nucleotide sequence complementary to nucleotide sequence SEQ ID NO 10 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 10, or an oligonucleotide having a nucleotide sequence homologous to the opposite reading direction nucleotide sequence:
SEQ ID NO 10.
cgaaccccac ctcc 14.

16. The oligonucleotide of claim 13, comprising a nucleotide sequences of SEQ ID No 5 or comprising a nucleotide sequence homologous thereto; or an oligonucleotide comprising a nucleotide sequence complementary to nucleotide sequence SEQ ID NO 5 or comprising a nucleotide sequence homologous to the complementary nucleotide sequence; or comprising, in reading direction 5′→3′, a nucleotide sequence having an opposite reading direction to nucleotide sequence SEQ ID NO 5, or comprising a nucleotide sequence homologous to the opposite reading direction nucleotide sequence:
SEQ ID NO 5
caaccccaat cgaacccc*w ccwyc vnd**d §nm#hwhy 38
wherein w represents a or t;
in represents a or c;
y represents c or t;
v represents a or c or g;
d represents a or g or t;
h represents a or c or t;
n represents a or c or g or t;
represents insert “c”;
** represents insert “t”;
§ represents insert “g”; and
# represents insert “c”.

17. The oligonucleotide of claim 16, comprising a nucleotide sequence of SEQ ID NO 11 or comprising a nucleotide sequence homologous thereto; or an oligonucleotide having a nucleotide sequence complementary to nucleotide sequence SEQ :ID NO 11 or an oligonucleotide comprising a nucleotide sequence homologous to the complementary nucleotide sequence; or an oligonucleotide comprising, in reading direction 5′→3′, a nucleotide sequence having an opposite reading direction to nucleotide sequence SEQ ID NO 11 or an oligonucleotide having a nucleotide sequence homologous to the opposite reading direction nucleotide sequence:
SEQ ID NO 11
caaccccaat cgaaccccwc cwycvnddnm hwhy 34,
wherein w represents a or t;
m represents a or c;
y represents c or t;
v represents a or c or g;
d represents a or g or t;
h represents a or c or t; and
n represents a or c or g or t.

18. The oligonucleotide of claim 1, having nucleotide sequence SEQ ID NO 15 or a oligonucleotide comprising a nucleotide sequence homologous thereto; or a oligonucleotide comprising a nucleotide sequence complementary to nucleotide sequence SEQ ID NO 15 or an oligonucleotide comprising a nucleotide sequence homologous to the complementary nucleotide sequence; or an oligonucleotide comprising, in reading direction 5′→3′, a nucleotide sequence having an opposite reading direction to nucleotide sequence SEQ ID NO 15 or an oligonucleotide comprising a nucleotide sequence homologous to the opposite reading direction nucleotide sequence:
SEQ ID NO 15
caaccccaat cgaacccc*a cctccccg**a §aa#attc 38
wherein represents insert “c”;
** represents insert “t”;
§ represents insert “g”; and
# represents insert “c”.

19. The oligonucleotide of claim 18, having SEQ ID NO 16 or an oligonucleotide having a nucleotide sequence homologous thereto; or an oligonucleotide having a nucleotide sequence complementary to nucleotide sequence SEQ ID NO 16 or an oligonucleotide comprising a nucleotide sequence homologous to the complementary nucleotide sequence; or an oligonucleotide having, in reading direction 5′→3′, a nucleotide sequence having an opposite reading direction to the nucleotide sequence SEQ ID NO 16 or an oligonucleotide having a nucleotide sequence homologous to the opposite reading direction nucleotide sequence:
SEQ ID NO 16
caaccccaat cgaaccccac ctccccgaaa attc 34.

20. The oligonucleotide of claim 13, having a nucleotide sequence of SEQ ID NO 3 or of SEQ ID 4 or of SEQ ID NO 10 or of SEQ ID NO 11, having one or more markings at one or at both of their 5′ ends and/or 3′ ends.

21. The oligonucleotide of claim 20, wherein the one or more markings are markers.

22. The oligonucleotide of claim 21, wherein the one or more markings are fluorescence markers or quenchers.

23. The oligonucleotide of claim 13, having a nucleotide sequence of SEQ ID NO 3 or of SEQ ID 4 or of SEQ ID NO 5 or of SEQ ID NO 10 or of SEQ ID NO 11 or of SEQ ID NO 14 or of SEQ ID NO 15 or of SEQ ID NO 16 in combination with one or more further oligonucleotides selected from forward primers and reverse primers.

24. The oligonucleotide of claim 13, having a nucleotide sequence of SEQ ID NO 3 or of SEQ ID 4 or of SEQ ID NO 5 or of SEQ ID NO 10 or of SEQ ID NO 11 or of SEQ ID NO 14 or of SEQ ID NO 15 or of SEQ ID NO 16, wherein a primer (1) comprises an oligonucleotide having a nucleotide sequence of SEQ ID NO 6 or an oligonucleotide having a nucleotide sequence homologous thereto;
or an oligonucleotide having a nucleotide sequence complementary to nucleotide sequence SEQ ID NO 6 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 6 or in the form of an oligonucleotide having a nucleotide sequence homologous to the opposite reading direction nucleotide sequence:
SEQ ID NO 6
gcactatatt acaccaaccc c 21
and/or wherein a primer (2) comprises an oligonucleotide having a nucleotide sequence of SEQ ID NO 7, or an oligonucleotide having a nucleotide sequence homologous thereto; or an oligonucleotide having a nucleotide sequence complementary to nucleotide sequence SEQ ID NO 7 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 7 or an oligonucleotides having a nucleotide sequence homologous to the opposite reading direction nucleotide sequence:
SEQ ID NO 7
catgcawctc mcccgtm 17,
wherein w represents a or t; and
m represents a or c.

25. The oligonucleotide of claim 24, having a nucleotide sequence of SEQ ID NO 12 or an oligonucleotide having a nucleotide sequence homologous thereto;
or an oligonucleotide having a nucleotide sequence complementary to nucleotide sequence SEQ ID NO 12 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 12 or an oligonucleotides having a nucleotide sequence homologous to the opposite reading direction nucleotide sequence:
SEQ ID NO 12
catgcatctc ccccgta 17.

26. The oligonucleotide of claim 24, wherein primer (1) is a forward primer and/or primer (2) is a reverse primer; or wherein primer (2) is a forward primer, and/or primer (1) is a reverse primer.

27. The oligonucleotide of claim 24, comprising as the primer (1) an oligonucleotide having a nucleotide sequence of SEQ ID NO 8 or an oligonucleotide having a nucleotide sequences homologous thereto; or an oligonucleotide having a nucleotide sequence complementary to nucleotide sequence SEQ ID NO 8 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 8 or an oligonucleotide having a nucleotide sequence homologous to the opposite reading direction nucleotide sequence:
SEQ ID NO 8
taaccaactg gcactatatt acaccaaccc caat 34
and/or comprising, as the primer (2), an oligonucleotide having a nucleotide sequence of SEQ ID NO 9 or an oligonucleotide having a nucleotide sequence homologous thereto; or an oligonucleotide having a nucleotide sequence complementary to nucleotide sequence SEQ ID NO 9 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 9 or an oligonucleotide having a nucleotide sequence homologous to the opposite reading direction nucleotide sequence:
SEQ ID NO 9
vbsbnrdwwk catgcawctc mcccgtmcat tatyt*bn 38
wherein s represents c or g;
w represents a or t;
r represents a or g;
y represents c or t;
k represents g or t;
v represents a or c or g;
d represents a or g or t;
b represents c or g or t;
n represents a or c or g or t; and
* represents insert “s”.

28. The oligonucleotide of claim 27, having the nucleotide sequence of SEQ ID NO 13 or an oligonucleotide having a nucleotide sequence homologous thereto; or an oligonucleotide having a nucleotide sequence complementary to nucleotide sequence SEQ ID NO 13 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 13 or an oligonucleotide having a nucleotide sequence homologous to the opposite reading direction nucleotide sequence:
SEQ ID NO 13
ggccagaatt catgcatctc ccccgtacat tattt*ta 38
wherein represents insert “s”.

29. The oligonucleotide of claim 27, wherein primer (1) is a forward primer and/or primer (2) is a reverse primer; or wherein primer (2) is a forward primer, and/or primer (1) is a reverse primer.

30. An oligonucleotide
(i) having sequence SEQ D NO 1 or an oligonucleotide having a nucleotide sequence homologous thereto; or
(ii) an oligonucleotide having a nucleotide sequence complementary to nucleotide sequence SEQ ID NO 1, or an oligonucleotide having a nucleotide sequence homologous to the complementary nucleotide sequence; or
(iii) an oligonucleotide having, in reading direction 5′→3′, a nucleotide sequence having an opposite reading direction to nucleotide sequence SEQ ID NO 1, or an oligonucleotide having nucleotide sequence homologous to the opposite reading direction nucleotide sequence:
SEQ ID NO 1
ccccwccwcc vnd 13,
wherein w represents a or t;
v represents a or c or g;
d represents a or g or t; and
n represents a or c or g or t.

31. The oligonucleotide of claim 30, comprising, or consisting of, a nucleotide sequence of SEQ ID NO 2, or an oligonucleotide having a nucleotide sequence homologous thereto; or an oligonucleotide having a nucleotide sequence complementary to nucleotide sequence SEQ ID NO 2 or an oligonucleotide having a nucleotide sequence homologous to the complementary nucleotide sequence; or an oligonucleotide having, in reading direction 5′→3′, a nucleotide sequence having an opposite reading direction to nucleotide sequence SEQ ID NO 2 or an oligonucleotide having nucleotide sequence homologous to the opposite reading direction nucleotide sequence:
SEQ ID NO 2
ccccacctcc ccg 13.

32. A method of amplifying a pathogen nucleotide sequences, preferably for the exclusive amplification of Trypanosoma cruzi DNA sequences in a mixture of DNA of various origin, particularly preferred by PCR, mostly preferred by real time PCR, or a method of determining the presence or absence of DNA of Trypanosoma cruzi in the body of an animal or of a human, preferably in the body of an insect, of a poultry, of a mammal or of a human, further preferred in a sample of a body tissue or of a body liquid of a poultry, of a mammal or of a human, most preferred in a sample of tissue or in a. sample of blood or in a sample of plasma or in a sample of serum of a human, or a method of diagnosing Chagas disease in the body of an animal or of a human, preferably in the body of an insect, of a poultry, of a mammal or of a human, further preferred in a sample of a body tissue or of a body liquid of a poultry, of a mammal or of a human, most preferred in a sample of tissue or in a sample of blood or in a sample of plasma or in a sample of serum of a human, wherein the method comprises employing the nucleotide of claim 13.
